(19) United States
(12) Patent Application Publication
(10) Pub. No.: US 2003/0152480 A1
(43) Pub. Date: Aug. 14, 2003

(54) Sanitizing mailbox apparatus and method

(76) Inventor: John C.K. Sham, Hong Kong (HK)

Correspondence Address: MICHAEL BEST & FRIEDRICH, LLP, 100 E WISCONSIN AVENUE, MILWAUKEE, WI, 53202, US

(21) Appl. No.: 10/072,452

(22) Filed: Feb. 8, 2002

(51) Int. Cl. A61L 2/14; A61L 2/00

(52) U.S. Cl. 422/28; 422/292; 422/300; 422/186.07; 422/1

(57) ABSTRACT

The present invention provides an article sanitation device preferably having a container in which articles to be sanitized can be received and a duct through which sanitizing fluid is moved into and/or out of the container. In some preferred embodiments, the sanitation device is part of a mailbox capable of de-contaminated mail placed therein. Preferably, ozone is employed as the sanitizing fluid, is created and neutralized by an ozone generator and an ozone neutralizer, and is moved with a fan through the various components of the article sanitation device. By employing a simple design, the cost of the sanitation device is relatively low. In addition, the use of ozone and an agitating article container in some embodiments helps to effectively and quickly sanitize all types, sizes, and numbers of articles.

### FIELD OF THE INVENTION

The invention relates to sanitation devices and methods, and more particularly to devices and methods for sanitizing items using sanitizing fluid.

### BACKGROUND

A number of different devices and methods exist for sanitizing articles of all types, including without limitation devices and methods for sanitizing clothing, medical instruments, food and drink, utensils, laboratory equipment, and other articles. The manner in which these devices and methods are employed to sanitize articles is at least partially dependent upon the type of articles sanitized and the type of contaminants to be sanitized. In some cases for example, the article to be sanitized is exposed to a gas or liquid fluid medium capable of decontaminating the article. In other cases, the article is exposed to light or other energy (such as infra-red, microwave, ultraviolet, and ultrasonic energy), chemicals, and the like for this purpose. Conventional sanitizing devices and methods are employed to destroy a wide variety of contaminants, including without limitation bacteria, viruses, spores, fungi, molds, and other organisms. Some sanitizing devices and methods are also or instead employed to sanitize articles from non-organic substances (e.g., chemicals and chemical compounds).

The cost of sanitizing equipment and/or the cost of the sanitizing process is often quite high. In many cases, the effectiveness of sanitizing devices or methods is commensurate with the cost of such devices or methods, forcing many consumers to do without sanitizing devices and methods that are needed to protect themselves and others.

For example, some sanitizing devices employ radiation in order to sanitize articles. Such devices are often used to sanitize fruits, meats, and other food products in order to kill bacteria and other microorganisms that could otherwise be harmful if the consumed. These types of devices typically include one or more radiation sources that emit radioactive waves, and therefore can require the use of high-density radiation-shielding materials (e.g., lead) in their construction. This type of large and heavy device construction increases the cost of radiating sterilizing devices, can significantly increase device complexity, and can present issues regarding potentially dangerous radiation emissions. Other types of conventional sanitizing devices and methods share similar problems regarding device complexity and cost.

As another example, some conventional sanitizing devices and methods employ a liquid medium which sanitizes articles through direct contact. Although suitable for sanitizing many types of articles, many other articles cannot be sanitized in this manner without damage to the articles. In other sanitizing devices and methods, non-liquid fluids (e.g., gases and gas compounds) are used for sanitizing articles. However, in these devices and in liquid sanitizing devices, design issues arise regarding flow control and containment of the sanitizing fluid and full exposure of articles to the sanitizing fluid.

Still other limitations of conventional sanitizing devices and methods exist. For example, many sanitizing devices and methods are capable of destroying a limited number of microorganism types. In addition, some sanitizing devices and methods require a significant exposure time to fully sterilize articles or are capable of fully sterilizing only a limited number of articles at one time. Also, the complexity of many conventional sanitizing designs results in expensive devices that are well suited for sale to the public at large.

In light of the problems and limitations of the prior art described above, a need exists for an article-sanitizing device and method that is relatively simple in construction, inexpensive, well-suited for simultaneously sanitizing multiple articles, capable of sanitizing articles of varying size, effective in destroying a number of different microorganisms, and can sanitize many different types of articles with no damage thereto. Each preferred embodiment of the present invention achieves one or more of these results.

### SUMMARY

The present invention provides an article sanitation device preferably having a container in which articles to be sanitized can be received and a duct through which sanitizing fluid is moved into and/or out of the container to de-contaminate articles in the container. In some preferred embodiments, the sanitation device is part of a mailbox capable of de-contaminated mail placed therein. As is well known in the art, the term “fluid” is understood to mean any element that has the capacity to flow. The term fluid therefore includes gas media such as gas elements, chemicals, and chemical compositions, liquid media in any form (including mists and atomized substances), vapors, and the like.

In some preferred embodiments, the duct connects an outlet of the container with an inlet of the container, and guides the sanitizing fluid though a fluid flow path in which cleaning elements, chemicals, and chemical compositions are added to the fluid during the de-contaminating process.

Preferably, ozone is employed as the sanitizing fluid in the present invention, and more preferably acts as a sterilizing fluid to destroy all organisms on the articles. In this regard, the term “sanitizing” as used here and in the appended claims is intended to refer to the process of at least destroying a substantial amount of organisms on articles, and includes the narrower definition of “sterilizing” (destroying all organisms or all organisms of a particular type). Depending upon a number of factors such as exposure time, concentration, and resistance of various organisms, ozone and many other de-contaminating elements, chemicals, and chemical compositions can be employed either as sanitizing or as sterilizing media.

In those embodiments employing ozone to sanitize articles in the container, the present invention preferably includes an ozone generator and an ozone neutralizer located in the fluid flow, such as in the duct connecting the inlet and outlet of the container. Preferably, a fan moves the ozone sanitizing fluid through the container, through the ozone neutralizer to remove ozone from the fluid flow, through or past the ozone generator to receive more ozone, and back into the container. When the article sanitation device of the present invention is stopped, the fan can continue operating for a period of time without operation of the ozone generator in order to purge fluid in the device using the ozone neutralizer.

If desired, a filter can be located in the path of fluid flow in order to capture contaminants destroyed by the ozone or other sanitizing element, chemical, or chemical composition used.

Although the present invention can be employed to sanitize articles without moving the container or otherwise agitating the articles therein, some highly preferred embodiments perform these functions to better expose the articles (and contaminants thereon) to the cleaning fluid. For example, the container can be driven and rotated by a motor connected thereto. The tumbling and agitation of articles in the rotating container facilitates improved de-contamination of the articles.

Some preferred embodiments of the present invention can employ a controller that automatically begins sanitizing operations on articles upon closure of a door to contain the articles within the container. In conjunction with one or more indicators, the controller can provide status information regarding the sanitation device, such as a low-battery warning, maintenance warnings, the operational state of the device, and the like.

By employing a simple design, the manufacturing and consumer cost of the sanitation device according to the present invention is relatively low. In addition, the use of ozone and an agitating article container helps to effectively and quickly sanitize all types, sizes, and numbers of articles (e.g., bundles of mail delivered to businesses or residences).

Other features and advantages of the invention will become apparent to those skilled in the art upon review of the following detailed description, claims, and drawings.

### BRIEF DESCRIPTION OF THE DRAWINGS

The present invention is further described with reference to the accompanying drawings, which show preferred embodiments of the present invention. However, it should be noted that the invention as disclosed in the accompanying drawings is illustrated by way of example only. The various elements and combinations of elements described below and illustrated in the drawings can be arranged and organized differently to result in embodiments which are still within the spirit and scope of the present invention.

In the drawings, wherein like reference numerals indicate like parts:

FIG. 1 is a schematic representation of an article sanitation device according to a preferred embodiment of the present invention;

FIG. 2 is a schematic representation of the article sanitation device illustrated in FIG. 1, shown during operation;

FIG. 3 is a perspective view of a mailbox including the article sanitation device illustrated in FIG. 1;

FIG. 4 is an exploded rear perspective view of the mailbox illustrated in FIG. 3;

FIG. 5 is a cross-sectional view of the ozone neutralizer of the article sanitation device illustrated in FIGS. 1-4, taken along line 5-5 of FIG. 1;

FIG. 6 is a cross-sectional view of the ozone generator of the article sanitation device illustrated in FIGS. 1-4, taken along line 6-6 in FIG. 1;

FIG. 7 is a perspective view of the ozone generator illustrated in FIGS. 1-4;

FIGS. 8a and 8b are a flowchart illustrating a preferred embodiment of an operating cycle of the device shown in FIGS. 3-7; and

FIG. 9 is a schematic illustration of an exemplary controller suitable for use in the device shown in FIGS. 3-7 and capable of executing the operating cycle illustrated in FIGS. 8a and 8b.

Before the various embodiments of the invention are described in detail, it is to be understood that the invention is not limited in its application to the details of construction and the arrangements of the components set forth in the following description or illustrated in the drawings. The invention is capable of other embodiments and of being practiced or being carried out in various ways. Also, it is understood that the phraseology and terminology used herein is for the purpose of description and should not be regarded as limiting. The use of “including” and “comprising” and variations thereof herein is meant to encompass the items listed thereafter and equivalents thereof as well as additional items. The use of “consisting of” and variations thereof herein is meant to encompass only the items listed thereafter.

### DETAILED DESCRIPTION

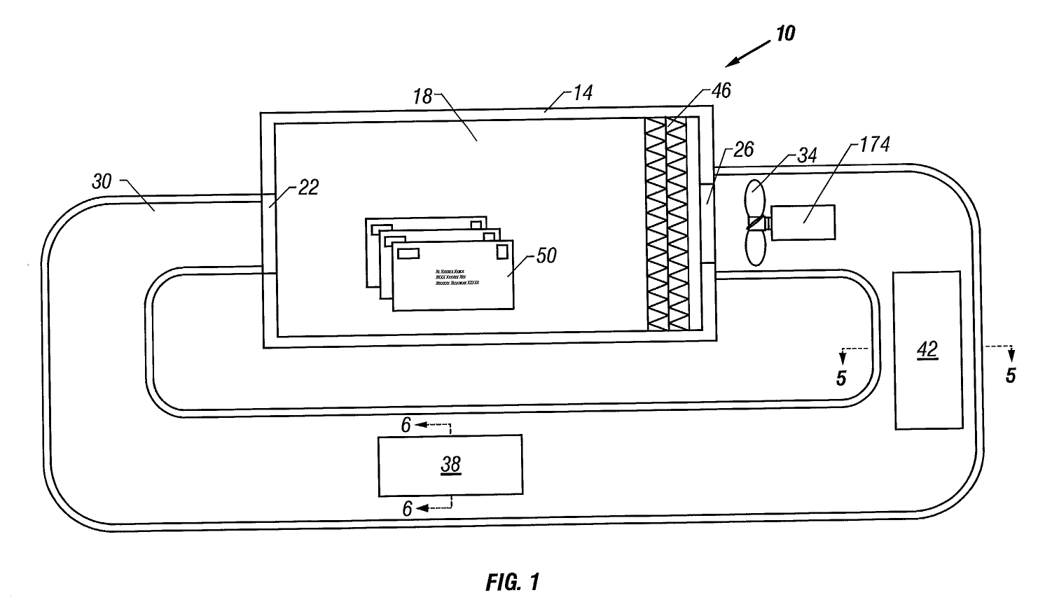

FIG. 1 illustrates an article sanitation device 10 including a container 14 that defines an inner chamber 18. In the illustrated preferred embodiment, the container 14 includes an inlet opening 22 and an outlet opening 26. Preferably, a duct 30 is in fluid communication with the inner chamber 18 and is connected at one end to the inlet opening 22 and at the other end to the outlet opening 26 such that the inner chamber 18 and the duct 30 cooperate to define a fluid flow path. The duct 30 is preferably defined by one or more pipes, conduits, tubes, hoses, or other elements capable of guiding fluid from the outlet opening 26 to the inlet opening 22 as will be described in greater detail below. As used herein and in the appended claims, the term “duct” therefore refers to any such element or elements. It should also be noted that the duct 30 can be defined in whole or in part by any of the elements and components of the article sanitation device 10 (which may or may not serve other purposes in the article sanitation device 10).

Preferably, the flow path generally extends from the inner chamber 18, through the outlet opening 26, the duct 30, and the inlet opening 22, and back into the inner chamber 18. A motorized fan 34 preferably communicates with the flow path to provide fluid flow through the flow path. The fan 34 can take any form desired, including without limitation propeller-type fans, centrifugal fans, and the like, and can be located anywhere along the duct 30 (or even within the container 14 in some embodiments). In some highly preferred embodiments however, the fan 34 is positioned inside the duct 30 adjacent the outlet 26.

With continued reference to FIG. 1 of the illustrated preferred embodiment, an ozone generator 38 preferably provides ozone (O₃) to the inner chamber 18 as described further below, and an ozone neutralizer 42 preferably neutralizes the ozone that flows out of the inner chamber 18. Although the present invention will operate to effectively sanitize articles without an ozone neutralizer 42, an ozone neutralizer 42 is preferred for purposes of user safety and environmental protection. In some preferred embodiments of the present invention, the ozone generator 38 is positioned upstream of the inlet 22 and the ozone neutralizer 42 is positioned upstream of the ozone generator 38. Such a configuration can provide a suitable amount of ozone to the inner chamber 18 for sanitation of articles placed therein. However, in other embodiments of the article sanitation device 10, the ozone generator 38 can be in different locations, such as adjacent to the inlet or outlet openings 22, 26 of the container 14 or even within the container 14. In the latter case and in which the container 14 is rotatable as described in greater detail below, power can be supplied as needed to the ozone generator 38 in any conventional manner (e.g., brush contacts providing power to the container 14). Similarly, other embodiments of the article sanitation device 10 can have the ozone neutralizer 42 in different locations, including any of those just described with reference to the ozone generator 38. In some highly preferred embodiments however, the ozone generator 38 is located between the chamber inlet 22 and the ozone neutralizer 42.

Although not required, the article sanitation device 10 can also include a filter element 46. The filter element 46 can take any conventional form (dependent at least partially upon the type of contaminants to be filtered), and in some preferred embodiments is a HEPA filter. The filter element 46 can be employed to remove particulate matter (e.g., live and dead microorganisms, dust, debris, and other matter) from the flow path in the article sanitation device 10. Preferably, the filter element 46 is positioned adjacent to the outlet 26 of the inner chamber 18. More specifically, the filter element 46 is preferably positioned between the inner chamber 18 and the other components located in the fluid path (e.g. the fan 34, the ozone neutralizer 42, and the ozone generator 38) to prevent contamination of those elements by matter to be filtered. Although the filter element 46 is preferably located adjacent to or immediately downstream of the chamber outlet 26 so that the filter element 46 can remove contaminants from articles immediately (or soon after) leaving the container 14, the filter element 46 can be located anywhere along the fluid path desired, and can even be located within the container 14.

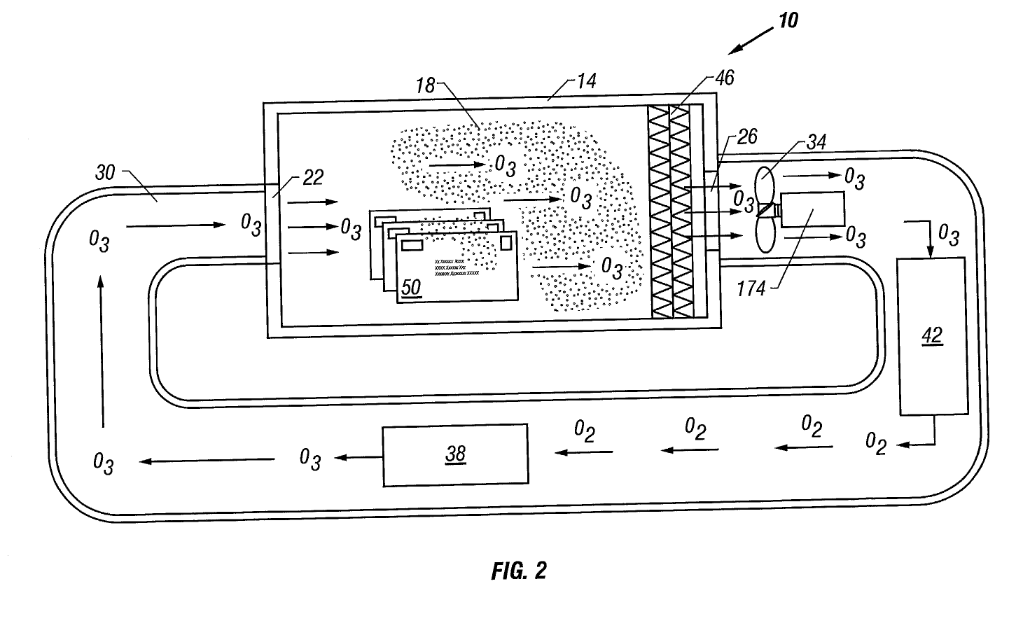

FIG. 2 illustrates the operation of the article sanitation device 10 shown in FIG. 1. An article 50 to be sanitized, such as an article of mail, a package, or a document, is inserted into the inner chamber 18. The fan 34 and the ozone generator 38 are activated and a stream of fluid flows along the flow path. Most preferably, the stream of fluid is non-liquid. As the fluid passes the ozone generator 38, additional amounts of ozone (O₃) are provided to the fluid (such as by an ionization process as discussed further below or in any other conventional manner). The fluid including the additional amount of ozone preferably flows along the duct 30 and into the inner chamber 18.

The articles 50 to be sanitized are preferably agitated within the container 14. Although this agitation can be generated in a number of different manners, agitation is preferably produced in the illustrated preferred embodiment by rotation of the inner chamber 18. Agitation of articles 50 in the container 14 is preferred in order to loosen and remove contaminants from the articles and in order to insure that an increased amount of article surface area is exposed to the ozone sanitizing medium flowing in the container 14. As the articles 50 are agitated, the ozone particles react with the particulate matter 54 and destroy biological contaminates that may be present on the articles 50. In other embodiments of the present invention, the articles 50 are not agitated in the container 14, and are instead at rest while being partially or fully immersed in the ozone sanitizing fluid.

As the fluid flow (which can contain contaminates and other matter from the articles 50 being sanitized), destroyed biological contaminates, and surplus ozone continues along the flow path, the fluid preferably encounters the filter element 46. Preferably, the filter element 46 removes the particulate matter 54 and destroyed biological contaminates from the flow path such that fluid exiting the filter 46 is free, substantially free, or at least has a reduced number of such particulate contaminates 54. In this regard, it should be noted that the filter 46 can be selected to filter any amount of such contaminates 54 from the fluid flow. Although the filter element 46 is preferably located adjacent to or immediately downstream of the container 14, the function performed by the filter element 46 can be performed in any other location along the flow path in the article sanitation device 10.

Preferably, the filtered fluid continues along the duct 30 toward the ozone neutralizer 42. Although the ozone neutralizer 42 can operate to convert ozone into other forms, the ozone neutralizer 42 preferably converts ozone into oxygen by a known process such as oxidization (discussed further below).

Next, the fluid preferably continues along the duct 30 and again reaches the ozone generator 38 where additional ozone is introduced into the flow path. The above-described cycle can be repeated for any amount of time desired, and is preferably repeated for an amount of time sufficient to destroy (and more preferably remove) all or substantially all contaminates introduced into the device 10 by the articles 50.

In some embodiments of the present invention, the ozone neutralizer 42 does not remove all of the ozone from the flow path in a single cycle. Therefore, the fan 34 in such embodiments can be controlled to continue moving fluid through the flow path after the ozone generator 38 is shut down in order to circulate fluid through the ozone neutralizer 42 one or more additional times. This delayed fan shutdown enables the device 10 to remove substantially all ozone present in the fluid and to prevent the release of ozone to the atmosphere when the device is opened to remove the articles 50.

The article sanitation device 10 can take a number of different forms for various applications. For example, the article sanitation device 10 can be a table or shelf-mounted unit, can be incorporated into a article handling device employed to perform functions upstream and downstream of the article sanitation device 10, and the like. Also, the article sanitation device 10 of the present invention can be employed to sanitize or sterilize any object, including without limitation food, utensils, clothing, medical instruments, and laboratory equipment. However, the inventors have found that the present invention provides excellent advantages and is extremely valuable in its application as a mailbox, and that its use to sanitize mail (e.g., documents, postcards, letters, packages, and the like) solves many problems encountered in mail handling.

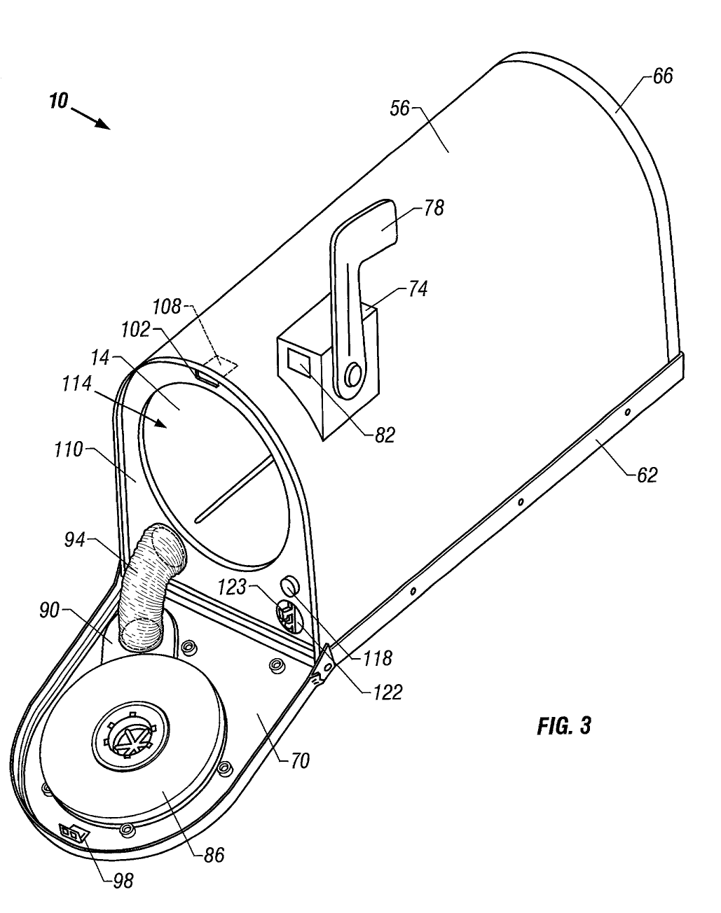

FIG. 3 is an example of the article sanitation device 10 of the present invention in the form of a mailbox. Preferably, the mailbox 10 includes an outer container or housing 58, a housing base 62 and end cap 66 (either or both of which can be part of the housing 58 or can be separate elements attached to the housing 58), and a lid or door 70. The particular elements and their orientations defining the exterior of the mailbox 10 can vary significantly based upon the type and shape of the mailbox, whether it is located in a row or bank of mailboxes, and upon other factors. Accordingly, the housing 58, base 62, and end cap 66 are described and illustrated herein by way of example only.

In the illustrated preferred embodiment, the door 70 is pivotally mounted to the housing base 62. In other embodiments, the door 70 can be mounted to the housing 58 or to any other portion of the mailbox 10 for pivotable, slidable, or any other movement desired, and can be permanently attached to the mailbox or can be removed (e.g., as a lid). Preferably, the housing 58 at least partially surrounds the container 14 and includes an operation indicator 74. In some embodiments, the operation indicator 74 includes a flag 78 for use in the traditional manner to indicate that mail for delivery has been placed in the mailbox 10. Preferably, the operation indicator 74 includes one or more indicia 82 that indicate the operating status of the mailbox 10 as will be described further below. The operation indicator 74 can be located on the top or side of the mailbox 10 for viewing by a user or other party when the door 70 is open or closed, can be in any other location such as on the door 70 or end cap 66, or can even be in a location disposed from the housing 58 and housing components 62, 66, 70.

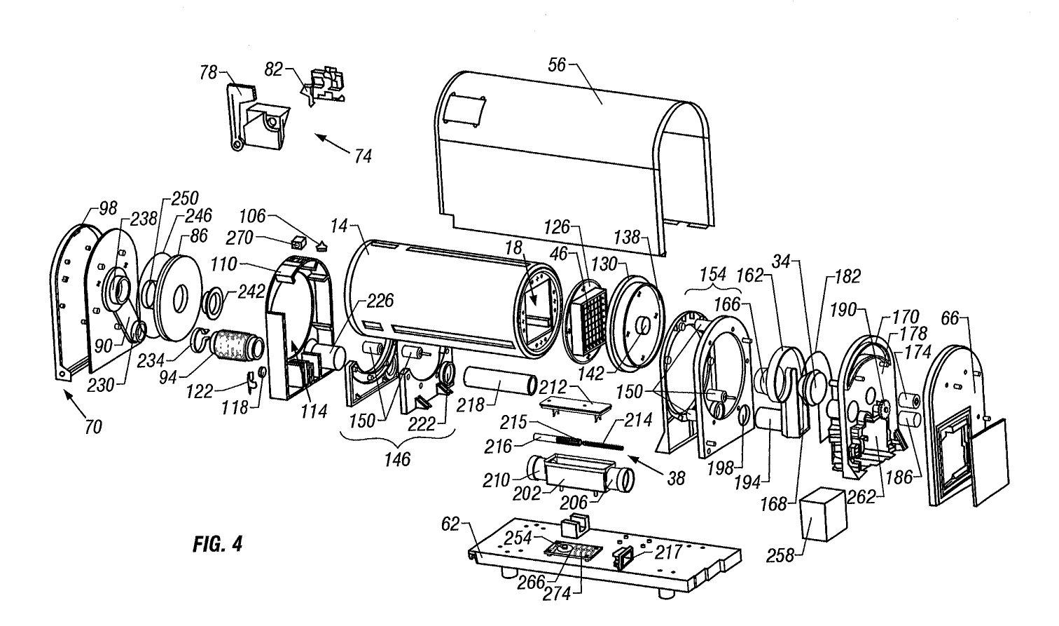

In some embodiments of the mailbox 10, the door 70 defines at least part of the duct 30 described above and/or provides a surface to which at least part of the duct 30 can be mounted. One example of such an arrangement is illustrated in FIGS. 3 and 4. In this illustrated embodiment, the duct 30 extends to the door 70, inside the door 70, and terminates at a location in fluid communication with the inner chamber 18 of the container 14. More specifically, a flexible hose 94 runs to the door 70 and is connected thereto in any conventional manner (such as by a collar, elastic band, hose clamp, crimp fit, adhesive or cohesive sealing material, threaded connection, compression fitting, or in any other manner used to connected ducts or pipes together and to other elements). In the illustrated preferred embodiment, the flexible hose 94 is coupled to a first flange 240 on the door 70 with a cable tie 234. A duct portion 90 defined in the body of the door 70 preferably runs from the flexible hose 94 to a location in fluid communication with the inner chamber 18 as mentioned above.

Some preferred embodiments of the present invention employ a plate 86 mounted to the door 70 in order to seal the container 14 when the door 70 is closed. This plate 86 can be stationary with respect to the container 14, in which case the container 14 preferably has a conventional seal that permits the container 14 to rotate with respect to the plate 86 while maintaining a sealing engagement with the plate 86. More preferably however, the plate 86 is mounted to rotate with the container 14, and therefore is preferably rotatably mounted to the door 70 as shown in FIGS. 3 and 4. In either type of embodiment, the duct 30 preferably extends through the plate 86 to establish fluid communication with the inner chamber 18. The duct 30 can extend through the plate 86 in any location, but most preferably extends therethrough in a central location as shown in the figures.

The door 70 and the plate 86 (if used) can seal the end of the container 14 in a number of different manners when the door 70 is closed. In the illustrated preferred embodiment for example, the housing 56 includes a face plate 110 that defines an access opening 114 to the inner chamber 18. The face plate 110 is preferably covered by the door 70 when the door 70 is closed, and can be pressed against by the plate 86 or door 70 to cover the access opening 114 and to seal the end of the container 14. In some preferred embodiments, the faceplate 110 includes a bypass button 118 and/or battery status indicators 122 and 123 (described in greater detail below).

With continued reference to FIG. 3, the door 70 preferably includes a second flange 238 that extends through the plate 86 and engages a securing ring 242 at the center of the plate 86. The securing ring 242 secures the rotating plate 86 to the door 70 while permitting relative rotation therebetween. A sealing ring, gasket, or other seal can be provided to help seal the inner chamber 18 when the door 70 is closed. In some preferred embodiments, a sealing ring 246, gasket, or other seal cooperates with an intermediate flange 250 to assist in substantially sealing the inner chamber 18 when the door 70 is closed.

Fluid preferably flows into the container 14 through the duct 30 as described above. However, one having ordinary skill in the art will appreciate that fluid can be introduced into the container 14 (whether mounted for rotation as described below or not) in a number of other manners. For example, the duct 30 can be a separate element extending between the door and the plate 86 for supplying fluid to the container 14. As another example, the flexible hose 94 can be eliminated in some embodiments which rely upon a mating fit between the duct portion 90 in the door 70 and the remainder of the duct 30 when the door 70 is closed. As yet another example, fluid can be supplied to a chamber between the door 70 and the end of the container 14 (covered by a plate 86 or otherwise) when the door is closed 70, thereby supplying this chamber with fluid which then enters the end of the container 14. In other embodiments, any part or all of the container 14 can have one or more apertures of any size and shape which permits fluid to enter around the container 14 and into the container 14 through the aperture(s). In this regard, any part or all of the entire container 14 can have apertures, such as apertures located only on a forward end, rear end, or middle of the container 14, one or more elongated slots running along the container 14, and the like. All such alternative manners of supplying fluid to the inside of the container 14 are possible and fall within the spirit and scope of the present invention.

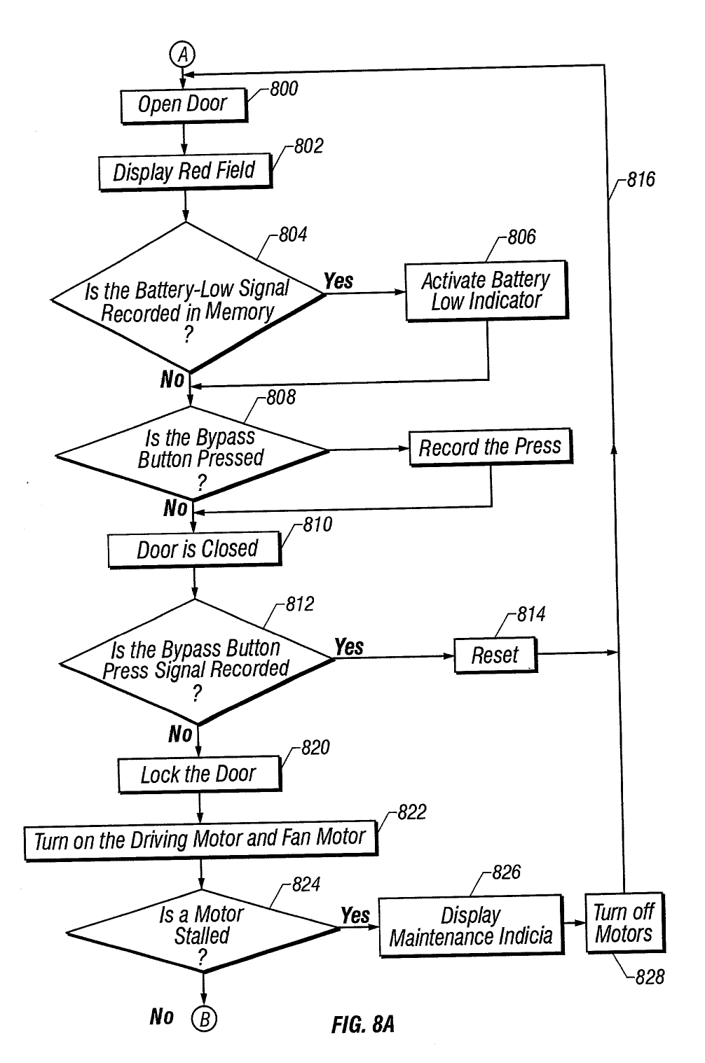

In the embodiment illustrated in FIGS. 3-9, decontamination or sanitizing of articles within and access to the interior of the mailbox 10 is controlled by a controller 254 (discussed below) that receives user input through a button, lever, switch, or other device located on the mailbox 10. In some embodiments, mailbox operation is dependent on status of the door 70 (i.e., open or closed) and is controlled by an electrically-actuated lock, latch, or similar device. Further, sanitizing can be started and/or stopped automatically by closure or opening of the door 70.

In the illustrated preferred embodiment for example, the door 70 includes a tab 98 that is received by a slot 102 having a micro-switch 106. Alternatively, the door 70 can have a post, pin, magnet, or other element engagable with the micro-switch 106 upon door closure. Closing the door 70 triggers the micro-switch 106, thereby providing an indication that the door 70 has been closed. The door closed indication is preferably transmitted via a communication link (that could, for example, take the form of a wire or wireless connection to the controller 254 to start sanitizing operations of the mailbox 10. As used herein and in the appended claims, the term “transmit” in its various forms refers to transmission of signals by wire or wirelessly. A number of alternative devices can be employed to automatically trigger sanitizing operations of the mailbox 10 when the door 70 is closed, including without limitation optical, magnetic, and other types of sensors.

Although operation of the mailbox 10 is preferably automatic through the use of the micro-switch 106 or other sensor responsive to the open or closed state of the door 70, the mailbox 10 can instead can be controlled (e.g., stopped and started) by user manipulation of a button, lever, switch, or other control preferably located on the mailbox 10.

With reference again to FIG. 4, the container 14 of the illustrated preferred embodiment includes a filter holder 126 and an end member 130. In this embodiment, the filter holder 126 includes a generally rectangular recess 134 that receives the filter element 46. The recess 134 can have any shape needed to receive the particular filter element 46 that will be used. In other embodiments, the filter element 46 can be positioned and retained in the container 14 in any other manner desired, including without limitation by clips, clamps, pins, and other fasteners, with adhesive or cohesive bonding material or tape, and the like. Also, the filter 14 can be located in other areas of the container 14, such as at the front of the container 14 for filtering fluid entering the container 14 or between the ends of the container 14. In other cases, the filter element 46 can be located outside of the container 14 for rotation therewith or can be stationary and retained in the fluid flow at any position upstream or downstream of the container 14 as described above.

The end member 130 preferably functions to close the end of the container 14 opposite the door 70 (with the exception of an aperture 142 permitting escape of fluid from the container 14), and can also be employed to drive the container 14 in embodiments of the present invention in which the container 14 is rotatable. The end member 130 can have any shape desired, and in the illustrated preferred embodiment is generally round. The end member 130 can be attached to the container in any conventional manner, such as with releasable fasteners permitting removal of the end member 130 in order to replace the filter element 46. Alternatively, the end member 130 can be permanently connected to or integral with the container 14. Other embodiments of the present invention do not have an end member 130, and instead permit exit of fluid from an open or partially open end of the container 14.

As mentioned above, the end member 130 can be employed as a driving element to turn the container 14 (if desired). In the illustrated preferred embodiment for example, the end member 130 has a set of internal gear teeth 138 which can be used to rotate the container 14 as will be described in greater detail below.

In those embodiments of the present invention employing a rotatable container 14 for agitating articles 50 placed therein, the container 14 is preferably rotatably supported on one end by a front bearing case 146 and on another end by a rear bearing case 154. The front and rear bearing cases 146, 154 can take any shape and form capable of supporting the container 14 (and preferably at least one bearing). For example, the bearing cases 146, 154 can be defined by frames, beams or arms, plates, or any other structure suitable for supporting the container 14. In the illustrated preferred embodiment for example, the bearing cases 146, 154 each have first and second sections connected together with fasteners or in any other conventional manner and supporting bearings (roller members 150). In some embodiments, either or both bearing cases (see rear bearing case 154 in FIG. 4) surround the container 14 and have an opening 158 sufficiently large to receive the container 14. The bearing cases 146, 154 are preferably mounted to the housing base 62, but can instead be secured within the mailbox 10 by being mounted to the housing 56 or to other structure within the housing 56.

Although two bearing cases 146, 154 are shown in the illustrated preferred embodiment, any number of bearing cases located anywhere along the container 14 can be employed as desired.

Preferably, fluid exits the container 14 through the end member 130 as described above. Also preferably, the aperture 142 of the end member 130 sealingly and rotatably engages an air drum 162 located downstream of the end member 130. The outlet of the end member 130 defining the aperture 142 can be sealingly and rotatably engaged with the air drum 162 in any conventional manner, such as by one or more seals or gaskets located on the end member 130 and/or on the air drum 162.

The air drum 162 preferably defines at least part of the duct 30, and can take any shape desired. In the illustrated preferred embodiment for example, the air drum 162 has a generally frusto-conical portion 166 to which the end member 130 is connected and a housing within which the fan 34 is at least partially received. Preferably, the fan 34 draws fluids through the aperture 142 and moves the fluid to the ozone neutralizer 42 (if used). The ozone neutralizer 42 can be in any location through which fluid moves in the mailbox 10, but in some preferred embodiments is located immediately downstream of the fan 34. In the illustrated preferred embodiment, the ozone neutralizer 42 is located within a chamber portion 168 of the air drum 162.

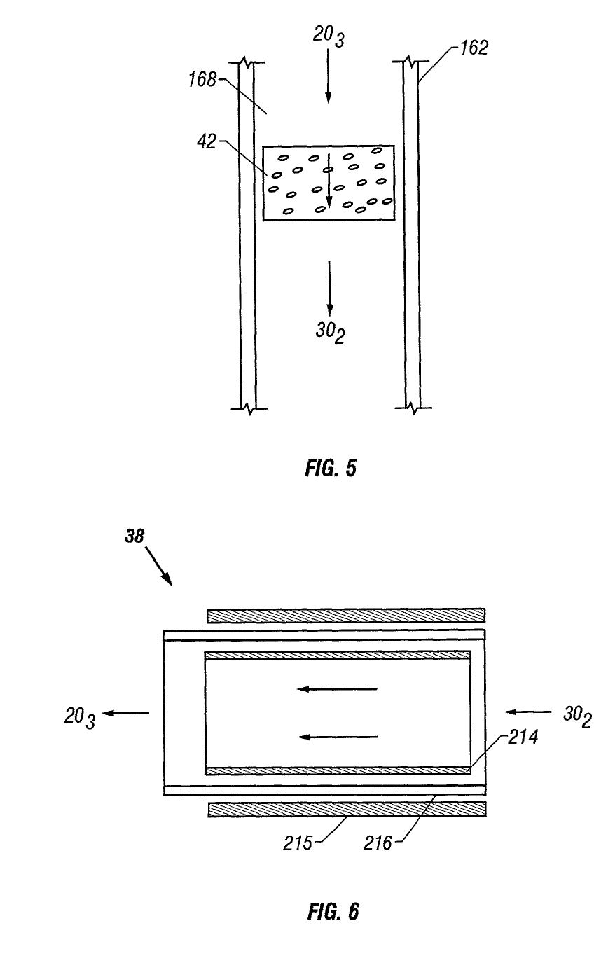

Referring now also to FIG. 5, in some embodiments the ozone neutralizer 42 comprises a mass of a catalytic chemical substance such as magnesium oxide. During operation, fluids flow through the magnesium oxide in order to convert the ozone in the fluids to oxygen through the following oxidation reaction (catalyzed by the magnesium oxide):

2O₃+Oxidization−>3O₂.

This chemical reaction is only one of several ways in which ozone in the fluid can be neutralized. One having ordinary skill in the art will recognize that a number of other manners exist for neutralizing ozone, including the use of other chemicals and chemical reactions. Each of these manners of ozone removal is considered to fall within the spirit and scope of the present invention.

As discussed in greater detail below, a number of other sanitizing elements, chemicals and chemical compounds can be employed as alternatives to ozone to sanitize and sterilize articles 50 in the present invention. In some cases, no neutralizing element or device is needed to neutralize the alternative elements, chemicals, or chemical compounds, such as in cases where the elements, chemicals, or chemical compounds deteriorate rapidly or are non-hazardous. In other cases however, a neutralizing element or device is needed, such as a filter, a containment tank or bath, a reaction chamber, and the like. One having ordinary skill in the art will appreciate that while such other elements, chemicals, and chemical reactions can be employed in much the same manner as the use of ozone (in the illustrated preferred embodiment) to sanitize and sterilize articles 50 in the container 14, such alternative elements, chemicals, and chemical compounds can require neutralization elements and devices that are different than the ozone neutralizer 42 described above. Accordingly, the present invention is not limited to the use of ozone in sterilizing and sanitizing articles 50, but includes other types of sanitizing and sterilizing elements, chemicals and chemical compounds along with their associated generating and neutralizing equipment.

With reference again to FIG. 4, the end cap 170 preferably functions to close the end of the housing 56, and in this regard can be integral with the housing 56. However, in some preferred embodiments such as that shown in the figures, the end cap 170 can also function to support a fan motor 174 (and/or the fan 34), a drive motor 186 used to drive the container 14, and other components of the mailbox 10. Like the bearing cases 146, 154, the end cap 170 can take any form desired. By way of example only, the end cap 170 in the illustrated preferred embodiment has inner and outer portions connected together with one or more conventional fasteners or in any other manner. Preferably, the end cap 170 is attached to the housing base 62 and the housing 56.

Like the door 70 described above, the end cap 170 can define part of the duct 30 through which fluid flows in the mailbox 10. For example, an end wall 178 of the end cap 170 can cooperate with the air drum 162 to define the chamber portion 168. If desired, a sealing member 182 such as a gasket or seal can be positioned between the air drum 162 and the end wall 178.

As mentioned above, a drive motor 186 is preferably employed to drive the container 14 (in embodiments having a movable container 14) and thereby to agitate articles 50 therein. The drive motor 186 is preferably supported by the end cap 170, and preferably has a drive gear 190 for directly or indirectly engaging with and driving the container 14. In those embodiments in which the drive motor 186 is separated from the container 14 by a wall (e.g., end wall 178) or other structure, the drive gear 190 and/or the shaft of the drive motor 186 preferably extend through such structure to directly or indirectly engage the container 14. In the illustrated preferred embodiment of the mailbox 10, the drive gear 190 engages the gear teeth 138 on the end member 130, thereby transmitting rotational motion from the drive gear 190 to the internal gear teeth 138 to rotate the container 14. One having ordinary skill in the art will appreciate that in other embodiments, the drive motor 186 can directly or indirectly drive the container 14 in different manners. In some embodiments, the drive motor 186 can be located in different areas of the mailbox 10 as needed. By way of example only, the drive motor 186 can drive a gear that meshes with teeth on the periphery of the container 14, a wheel that frictionally engages the end member 130 or a surface of the container 14, a sprocket that engages with the container 14, a belt, cable, or other flexible element trained about the container 14, or an axle extending from the container 14. All such alternative manners of driving the container 14 with the drive motor 186 fall within the spirit and scope of the present invention.

The air drum 162 preferably has an outlet through which fluid can pass to the ozone generator 38. The outlet can be an aperture in a wall of the air drum 162, can be an extension 194 of the air drum 162 as shown in FIG. 4, or can take any other form desired. If necessary, and depending upon the location of the ozone generator and other structure within the housing 56, apertures in such structure can be provided in order to connect the air drum 162 with the ozone generator 38. For example, the extension 194 of the air drum 162 in FIG. 4 preferably passes through an aperture 198 in the rear bearing case 154.

Preferably, the extension 194 of the air drum 162 is connected to a housing 202 of the ozone generator 38. Alternatively, the air drum 162 can be connected to the ozone generator housing 202 via one or more ducts. In either case, the connection of the air drum 162 to the ozone generator 38 can take any form suitable for connecting fluid ducts together, including those mentioned above with regard to the connection of the flexible hose 94 to the door 70.

Preferably, the ozone generator 38 is mounted to the housing base 62 (or to other housing structure of the mailbox 10), and includes an inlet 206 and an outlet 210. The housing 202 preferably houses the various components of the ozone generator 38, and can include a removable cover 212 to facilitate service or replacement of the ozone generator 38 in whole or in part.

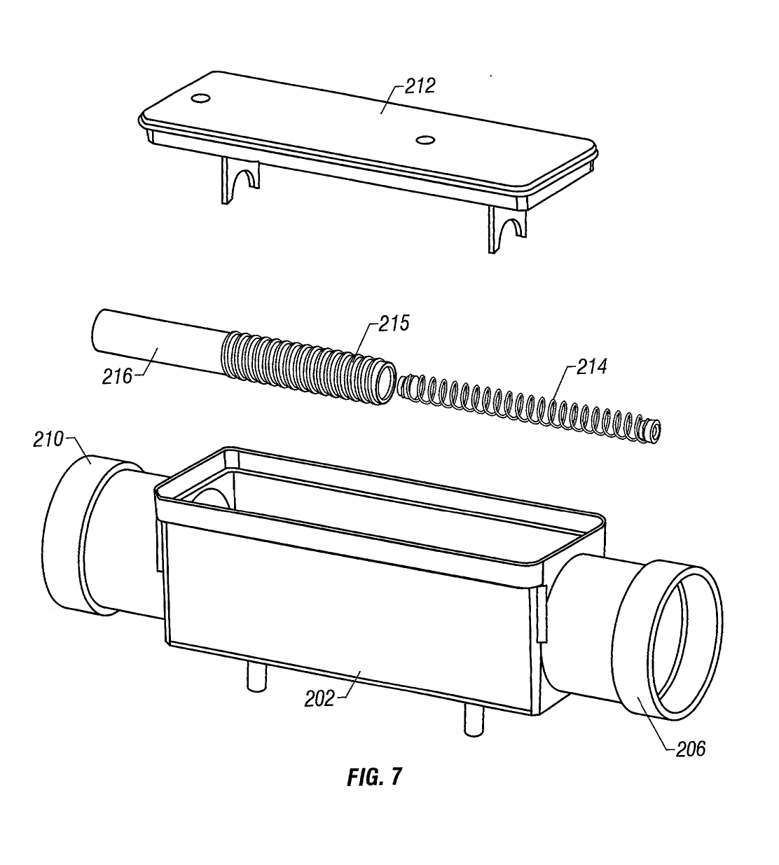

FIG. 6 provides a schematic illustration of the ozone generator 38 used in the preferred embodiment of FIGS. 1-5. The ozone generator 38 preferably includes a high voltage electrode 214 and a neutral electrode 215 surrounding the high voltage electrode 214. An insulator 216 is preferably positioned in the space between the high voltage electrode 214 and the neutral electrode 215. As illustrated, the space between the electrodes 214, 215 is a generally annular space due to the tubular configurations of the electrodes 214, 215. For example, the electrodes 214, 215 can each be configured as helically coiled stainless steel wires (the neutral electrode 215 being only partially shown in FIG. 6), with the insulator 216 configured as a ceramic tube having an aluminum coating on its outer surface. However, other shapes and arrangements of electrodes can instead be employed, such as flat plate electrodes. In some preferred embodiments such as the illustrated preferred embodiment, the high voltage electrode 214 receives an input voltage of approximately 3000 VAC, which is provided by a transformer 217 as discussed further below.

The electrically operated ozone generator 38 described above and illustrated in FIG. 6 is highly preferred for its relatively low cost and efficient operation. However, it will be appreciated by one having ordinary skill in the art that a number of other ozone generators can instead be utilized in the present invention. For example, other types of electrical ozone generators can utilize different types, materials, and configurations of electrodes 214, 215 and insulators 216. Other types of ozone generators can instead be employed, such as generators creating ozone via chemical reactions not requiring electrical input. Each of these alternative ozone generators falls within the spirit and scope of the present invention.

With reference again to the ozone generator 38 used in the illustrated preferred embodiment, the arrangement and configuration of the high voltage electrode 214, the insulator 217, and the neutral electrode 215 creates high-energy electrical discharges that results in the separation of oxygen molecules (O₂) into oxygen atoms (O₁). The unstable oxygen atoms then recombine with existing oxygen molecules to form ozone (O₃). This process of ozone creation is generally well known in the art as corona discharge ozone generation. A variety of other techniques and devices for ozone generation are well known in the art (as mentioned above) and are commercially available from a variety of sources. The above-described ozone generator 38 has been included as one example of a suitable ozone generation device. Accordingly, a variety of other ozone generating devices and techniques are suitable for use with the present invention.

As mentioned above, other sanitizing fluids can instead be employed to sanitize the articles 50 in the container 14. Such sanitizing fluids can include chlorine, iodine, iodophors, chlorohexidine, phenols, quaternary ammonium compounds, and aldehydes. Although any of these chemicals can be employed to sterilize and sanitize articles 50, ozone is most preferred due to its reduced corrosiveness, relatively low danger to users, and capacity for use in non-liquid form. Although a number of alternative sterilizing and sanitizing elements, chemicals and chemical compounds can damage some articles because they are effective only in liquid form (and must therefore be deposited as a mist or vapor upon the articles), such elements, chemicals and chemical compounds can be used as needed or desired. In some cases, an atomizer, diffuser, mister, or other device can be used to introduce sanitizing and sterilizing elements, chemicals, and chemical compounds into the fluid flow of the mailbox 10.

Referring again to FIG. 4, the outlet 210 of the ozone generator housing 202 is preferably coupled to a connecting pipe 218. The connecting pipe 218 defines a part of the duct 30, and is preferably directly or indirectly connected to the flexible hose 94 described above. To establish this connection, one or more apertures can be provided in structure located between the ozone generator 38 and the flexible hose 94. In the illustrated preferred embodiment for example, an aperture 222 is preferably provided in the front bearing case 146 through which the connecting pipe 218 extends. The connecting pipe 218 can extend to and directly connect with the flexible hose 94, or can instead connect to one or more other ducts leading to the flexible hose 94. With reference again to the illustrated preferred embodiment, the connecting pipe 218 is preferably connected to a rearwardly-extending flange 226 provided on the faceplate 110 in any conventional manner, such as those described above with reference to the connection between the flexible hose 94 and the door 70. In this manner, fluid can be transmitted from the ozone generator 38 to the duct portion 90 of the door 70 and to the container 14.

As discussed above, the duct 30 can be defined by and formed from a plurality of mailbox 10 components. In the illustrated preferred embodiment, the duct 30 extends from the aperture 142 formed in the end member 130, through the air drum 162, the ozone generator housing 202, the connecting pipe 218, the flexible tubing 94, and the duct portion 90 of the door 70, and substantially ends at the securing ring 242. Thus, fluid flow generated by the fan 34 circulates through the inner chamber 18 and through each component of the duct 30 when the fan 34 is rotatably driven by the fan motor 174.

The controller 254 preferably controls operation of the mailbox 10. The controller 254 can take a number of different forms, including without limitation electronics having one or more microprocessors, electronics having discrete logic elements, and any other conventional circuitry capable of controlling various electrical devices. The controller 254 can be mounted anywhere within or outside of the mailbox 10, but is preferably mounted to the housing base 62. The circuitry 254 preferably electrically communicates with various electrical components of the mailbox 10 to receive and send signals corresponding to various operating steps of the mailbox 10.

In some embodiments, the mailbox 10 includes a rechargeable battery 258 permanently or releasably connected to the controller 254 and utilized to supply power to the controller 254 and the various electrical components of the mailbox (including the transformer 217). Like the controller 254, the rechargeable battery 258 can be located anywhere inside or outside of the mailbox 10. In some preferred embodiments however, the rechargeable battery 258 is housed in a recessed portion 262 of the end cap 66, and is accessible for service, removal, and replacement through an access panel or door in the end cap 66, housing 56, or housing base 62.

The transformer 217 preferably converts the electrical output of the battery 258 to the high voltage electrical supply utilized by the ozone generator 38. It will be appreciated that some embodiments of the present invention can utilize alternative power supplies connected to the controller 254, including non-rechargeable batteries, AC power delivered by a conventional power line, solar panels (preferably cooperating with rechargeable batteries), and other types and combinations of power supplies.

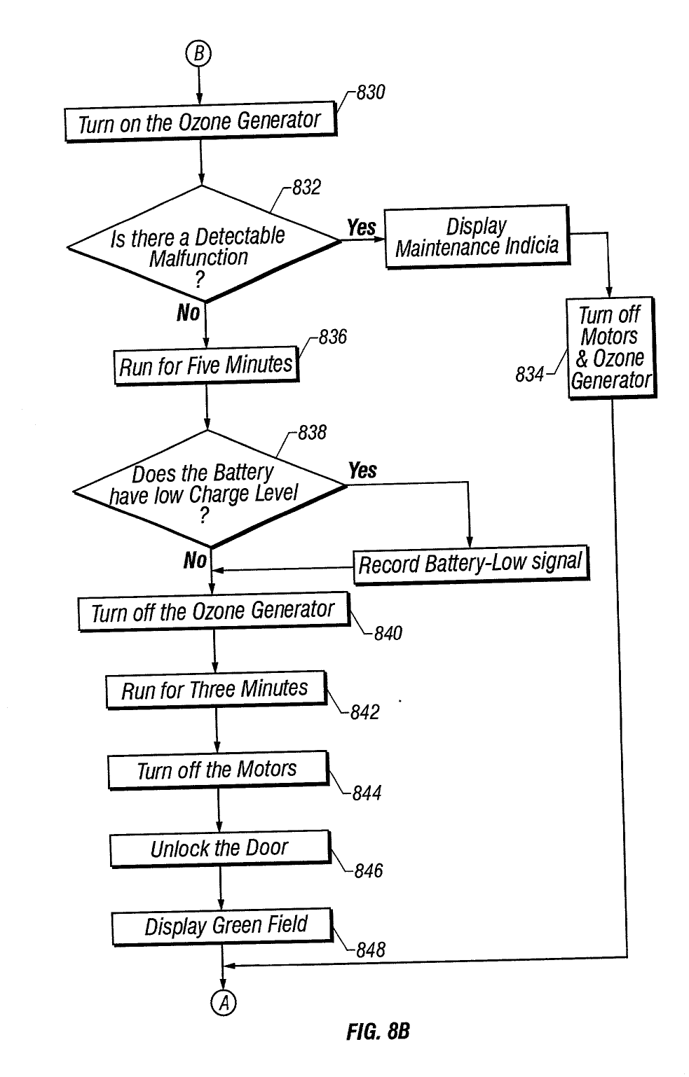

With continued reference to FIG. 4 and with further reference to FIGS. 8a and 8b, some embodiments of the controller 254 include a battery monitor 266 that monitors the charge level of the rechargeable battery 258. When the battery's charge reaches a predetermined low limit, the controller 254 preferably transmits a battery-low signal to the status indicators 122, 123. As mentioned above, the status indicators 122, 123 are preferably located on the face plate 110, and so are visible when the door 70 is open. Therefore, when a user opens the door 70 to insert and sterilize or sanitize articles 50, the status indicators 122, 123 are visible to indicate whether the battery 258 is low. In other embodiments, the status indicators 122, 123 can be located in other areas of the mailbox 10, such as on the exterior of the housing 56, on the end cap 66, and the like. In either case, the battery status indicators 122, 123 can indicate a low-battery condition at all times or when triggered to “wake up” (e.g., to show a low-battery condition only upon opening the door 70). In the latter case, the micro-switch 106, sensor, or other device used to transmit a signal to the controller 254 when the door 70 is detected in an open position preferably triggers the battery status indicators 122, 123 either directly or via the controller 254.

The battery status indicators 122, 123 can include a visual indicator 122, such as a blinking or steady light source having any color or changing colors, and can also or instead include an audio indicator 123, such as a constant or intermittent buzzer, beep, or other tone emitted in any conventional manner. It will be appreciated by one having ordinary skill in the art that any appropriate signal can be provided to indicate that the battery 258 has reached a low level of charge.

As discussed above, some embodiments of the present invention have an operation indicator 74. Preferably, the operation indicator 74 is connected to the controller 254 and receives signals from the controller 254 regarding at least one state of operation of the mailbox 10 directly or indirectly monitored by the controller 254. The operation indicator 74 can provide an operational status of the mailbox 10 at all times or when triggered to “wake up”, such as when the controller 254 receives a signal indicating that the door 70 has been opened as described in greater detail above. Like the battery status indicators 122, 123, the operational indicator 74 can take any conventional form, including without limitation one or more indicator lights of any color or changing color, audio signals generated in any conventional manner, and displays such as LCD, electro-luminescent, and LED displays.

In some preferred embodiments of the present invention, upon power up, the controller 254 clears all random access memory and determines whether the door 70 is open (step 800). If the door 70 is open, the controller 254 preferably enters a standby mode. If the door 70 is closed, the controller 254 performs certain other operations discussed below. It should be understood that the controller 254 is preferably event driven, meaning that the occurrence of certain events (such as opening and closing of the door 70) cause the controller 254 to execute certain operations. In the text that follows, the controller 254 is sometimes described in a temporal context. However, the use of temporal context is mainly for purposes of simplifying the description herein and not necessarily as a general indication that events are time driven or required to occur in a certain sequence.

In some preferred embodiments of the present invention, the micro-switch 106 or other sensor detects when the door 70 is opened by transmitting one or more signals directly to the operational indicator 74 or (more preferably) to the controller 254. In the latter case, the controller responds by transmitting a signal to the operational indicator 74. In response to such signal(s), the indicator 74 preferably indicates that the mailbox 10 is ready for operation (step 802). By way of example, an indicator light can turn red, an LED display can read “ready”, or a short beep can be generated upon opening of the door 70. Any suitable color or other indicia may be used to indicate that the door 70 is open. Alternatively, such an indicator can be generated in response to user manipulation of a control on or associated with the mailbox 10 and connected to the controller 254. By way of example only, this control can be a start, status indicator, or other control button (not shown).

As described above, the battery status indicators 122, 123 can also or instead be triggered to indicate whether the battery is low. Once the door 70 is opened in some embodiments, the controller 254 checks low battery signal data stored in memory (during the occurrence of a sanitizing or decontamination cycle) associated with the controller 254, as shown at step 804. If a low battery voltage condition is detected, the controller 254 preferably activates the battery status indicators 122, 123 accordingly as shown in step 806.

As shown at step 808, the controller 254 then preferably checks to see if the bypass button 118 has been pressed. If so, that event is preferably stored in memory. At step 810, the controller 254 checks for a change in the open/closed status of the door 70. No further action is taken by the controller until the door 70 is closed. As shown in steps 812 and 814, if the door is closed, but a bypass input has been recorded, the controller 254 clears the bypass record from memory. Control is then returned to step 800, as shown by loop 816.

To operate the mailbox to sanitize or sterilize articles 50 (e.g., mail), the articles 50 are preferably inserted into the mailbox 10 and the door 70 is closed. If the door 70 is closed, but no bypass input has been received, it is assumed that unsanitized mail 50 has been inserted into the mailbox 10. Preferably, the controller 254 receives one or more signals from the micro-switch 106 or other sensor indicating that the door 70 has been closed, and automatically responds by starting operation of the mailbox 10. Therefore, no further action is needed by the user.

If, however, mail 50 is being removed from the mailbox or if it is not desired to sanitize the articles 50 inserted into the mailbox 10, some preferred embodiments of the present invention are provided with a bypass button 118 as mentioned above. This bypass control can take a number of other forms, including without limitation a switch, lever, dial, or other control. Preferably, user manipulation of the bypass button 118 causes one or more signals to be transmitted to the controller 254 in order to prevent mailbox operation upon closure of the door 70. In some embodiments for example, the controller 254 stores the bypass signal(s) such that upon closing the door 70, no further actions are performed by the mailbox 10. In other embodiments, mailbox operation only commences upon user manipulation of a control connected to the controller 254, such as a start button that can be depressed after the door 70 has been closed.

With reference again to the illustrated preferred embodiment, if the bypass button 118 is not pressed and the door 70 is closed, the controller 254 preferably provides one or more electrical signals to an electromagnet 270 positioned near the slot 102 (see FIG. 4). The electromagnet 270 preferably responds by locking the door 70 in the closed position (step 820) until one or more signals are received from the controller 254 or from other devices coupled thereto to unlock the door 70. In other embodiments, the controller 254 can transmit one or more signals to any other type of locking device, such as a latch driven in any conventional manner, a solenoid having an armature that extends to lock the door 70, an automotive-style power lock assembly, and the like. Any type of locking device capable of being driven by the controller 254 can instead be used.

To begin the sterilizing or sanitizing process, signals are generated to activate the fan motor 174 and the drive motor 186 (if the container 14 is driven to agitate articles 50 during operation of the mailbox 10) as indicated at step 822. Preferably, these signals are generated by the controller 254. If, for any reason, either motor 174, 186 becomes stalled (step 824), the controller 254 will preferably provide one or more signals to the operational indicator 74 (step 826) to generate a maintenance-required indicia and will shut off the motors 174, 186 (step 828). Such indicia can take any form desired, including those described above with reference to the operational indicator 74. For example, the operational indicator 74 can display an X, can flash red, can generate a sustained or repeated beep, buzz, or other tone to indicate that maintenance is needed.

If the motors 174, 186 instead begin rotating normally, the controller 254 preferably transmits one or more signals to activate the ozone generator 38 (step 830) and to turn on the fan 34. Also preferably, the operational indicator 74 indicates proper mailbox operation in any manner desired. In the illustrated preferred embodiment for example, the operational indicator can display a continuous color (such as red), beep occasionally, or display a word such as “sanitizing”.

In some preferred embodiments, the controller 254 performs a partial or full self-diagnosis prior to, during, or after operation of the mailbox 10. For example, the controller 254 in the illustrated preferred embodiment preferably performs a general diagnostic check for general malfunctions after the ozone generator 38 has been activated (step 832). If any malfunctions are discovered (or if selected malfunctions are discovered), the ozone generator 38 and the motors 174, 186 can be shut down by the controller 254 (step 834), in which case the operational indicator 74 preferably displays the maintenance-required indicia.

If no malfunctions are discovered, the ozone generator 38 and the motors 174, 186 are preferably run for a desired amount of time (step 836). In various embodiments of the present invention, this time is factory-set or is user-adjustable via a user-manipulatable control connected to the controller 254. In the illustrated preferred embodiment, the motors 174, 186 are run for a cleaning period of about 5 minutes while the ozone generator 38 produces ozone. A timer 274 connected to or part of the controller 254 is preferably provided in order to count the length of time of the cleaning period. It will be appreciated that the length of the sanitizing or sterilizing process can vary based at least partially upon the type and number of articles 50 being sanitized or sterilized, the amount of production of the ozone generator 38, and other factors. During the cleaning period, fluid (e.g. air) is provided with increased amounts of ozone and is circulated through the inner chamber 18 and the duct 30. Simultaneously, the articles 50 can be agitated by rotation, vibration, or other movement of the container 14 or by other types of agitation described in greater detail below.

As the cleaning fluid is passed through the articles 50 (and as the articles 50 are agitated in some embodiments) contaminants in and on the articles 50 are exposed to the fluid and ozone flowing through the inner chamber 18. Article tumbling, turning, and other agitation assists in exposing the various surfaces of the articles 50 during the cleaning process. The ozone provided by the ozone generator 38 preferably reacts with the contaminates and kills microorganisms present in the container 14. Preferably, the carcasses of the destroyed contaminates as well as other particulate matter are then filtered from the fluid flow by the filter 46.

The sanitized, filtered fluid is then preferably conducted through the air drum 162 and past the ozone neutralizer 42 where at least some of the ozone in the fluid is neutralized into oxygen (O₂) by the previously-described oxidation reaction. The fluid then preferably continues to the ozone generator 38 where additional amounts of ozone are added to the fluid. Finally, the fluid flows through the connecting pipe 218, the flexible tube 94, through the duct portion 90 of the door 70, and back into the inner chamber 18 where additional contaminates are destroyed by the ozone and are filtered from the fluid by the filter 46.

Once the cleaning period has expired (e.g. after 5 minutes or another length of time), the controller 254 preferably transmits one or more signals to turn off the ozone generator 38 (step 840). Once the ozone generator 38 is turned off, a purging period begins (step 842) in which the ozone neutralizer 42 removes remaining ozone from the fluid flow. Preferably, during the purging period, the fan motor 174 continues to run such that fluid is circulated through the inner chamber 18 and the duct 30. Because the ozone generator 38 is off, additional ozone is not added to the fluid flow and the ozone neutralizer 42 is able to remove the ozone or substantially all of the ozone from the fluid.

In some embodiments, the purging period lasts approximately 3 minutes, although the exact length of the purging period may vary based upon the size of the duct 30 and inner chamber 18, the amount of ozone therein, the power of the fan 34, and other factors. Although the ozone generator 38 is preferably turned off prior to the fan 34 in order to provide for a purging period as just described, the ozone generator 38 and fan 34 can be turned off simultaneously if desired (in which case no purging period exists). Also, in those embodiments of the present invention in which the articles 50 are agitated, the drive motor 186 can be turned off with the ozone generator 38, with the fan 34, or at any other time desired.

After the purging period has expired (in those embodiments of the present invention employing a purging period), the controller 254 preferably transmits one or more signals to turn off the motors 174, 186 (step 844), to unlock the door 70 (step 846), and to indicate that the cleaning process has been completed via the operational indicator 74 (step 848). The cleaning-complete indicia can be a color displayed by the operational indicator 74 (e.g., a green LED), one or more chimes, beeps, or other tones generated in any conventional manner, or a word such as “complete” displayed by the operational indicator 74. With the door 70 unlocked and the cleaning-complete indicia displayed, the door 70 can be opened and the articles 50 can be removed from the inner chamber 18.

In those embodiments of the present invention having a bypass button 118, the bypass button 118 is preferably pressed prior to re-closing the door 70 in order to prevent commencement of another cleaning period. At any time during the cleaning process, such as after running the ozone generator 38 and motors 174, 186 for a period of time or after a purging period, battery power can be checked to determine if a low battery level exists (step 838). This check can be performed in any conventional manner, and in the illustrated preferred embodiment is performed by the controller 254. In the event a low battery level is detected, one or more signals are transmitted to the controller 254 to be saved in memory for recall after the door 70 is opened and/or after the cleaning operation is completed. Alternatively, such signals can be transmitted to the controller 254 or directly to the battery status indicators 122, 123 to immediately indicate such a condition.

The embodiments described above and illustrated in the figures are presented by way of example only and are not intended as a limitation upon the concepts and principles of the present invention. As such, it will be appreciated by one having ordinary skill in the art that various changes in the elements and their configuration and arrangement are possible without departing from the spirit and scope of the present invention as set forth in the appended claims. For example, the direction of fluid flow illustrated in the figures is preferred but is not required. Specifically, the direction of fluid flow can be reversed in the embodiments described above and illustrated in the figures while still operating to sterilize or sanitize articles 50. In such cases however, the locations of the ozone generator 38 and the ozone neutralizer 42 are preferably reversed so that the ozone generator 38 is located downstream of the ozone neutralizer 42 but upstream of the container 14.

As another example, it should be noted that a container 14 having an inlet 22 and a separate outlet 26 is not required to practice the present invention. In other embodiments, fluid can be drawn or pushed into the container 14 using a fan 34 or any other type of fluid moving device, the articles 50 in the container can be exposed to such fluid for a period of time, and the fluid can be pushed or drawn out of the container 14 using the same aperture used to move the fluid into the container 14. In other embodiments (particularly in cases where purging of the sanitizing or sterilizing fluid is not needed or is less of a concern), fluid can be drawn or forced into the container 14 for cleaning the articles 50 therein for a period of time after which the door 70 can be opened and the articles 50 removed. Still other embodiments of the present invention exist having a single container aperture, each one of which falls within the spirit and scope of the present invention.

A number of embodiments described above and illustrated in the figures employ a container 14 that can be driven to rotate and to thereby agitate articles 50 therein during the sanitizing or sterilizing process. Although this manner of article agitation is preferred, it should be noted that other types of container movement can be employed to agitate the articles 50.

In other embodiments for example, the container 14 can be connected to any conventional vibrating device, such as pneumatic, hydraulic, or solenoid-driven actuators, rotational vibrators, and the like that impart vibration to the container 14 and to articles 50 therein. Alternatively, the container can be connected to attached to any type of reciprocating driver in order to perform this same function using larger container and article displacements. In still other embodiments, one or more elements located within the container 14 can be driven to agitate articles 50 therein without necessarily moving the container 14. Examples of such agitating elements include one or more augers, paddles, fingers, or arms located within or extending inside the container 14 and rotated by one or more motors connected thereto or otherwise actuated to move by one or more pistons, solenoids, hydraulic or pneumatic actuators, and the like connected thereto.

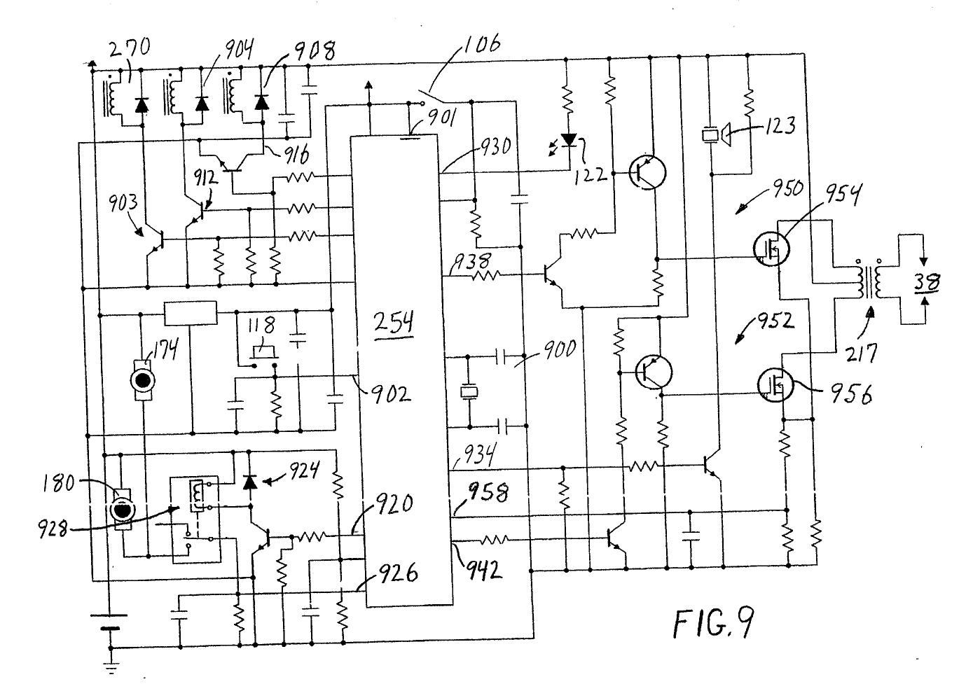

Further details of the controller 254 and other electrical components for one exemplary embodiment are provided in FIG. 9. The controller 254 may take the form of a programmable device such as a microprocessor or microcontroller. In the embodiment shown, the controller 254 receives a clock signal from an oscillator 900. The controller 254 receives input from the door switch 106 and the bypass switch 118. The door switch 106 is coupled to a memory clear or reset pin 901. The bypass switch 118 is coupled to pin 902. Input received through pin 902 changes the bypass flag in the software executed by the controller 254.

As discussed above, upon the occurrence of certain conditions, the controller 254 will lock the door 70 by actuating the solenoid or electromagnet 270 through a drive circuit 903. The operation indicator 74, in the form shown, is controller by two solenoids 904 and 908, which are driven by two drive circuits 912 and 916, respectively. The solenoids 904 and 908 drive mechanisms that rotate or otherwise change indicia presented by the operation indicator 74.

The fan motor 174 and drive motor 180 receive a drive signal (e.g., a logic high) through an output pin 920. The drive signal is conditioned in motor drive circuit 924 and then delivered to the motors 174 and 180, which in the embodiment shown are connected in parallel circuit paths. Operation of the motor is monitored through pin 926, which is coupled to relay 928. If the controller senses a trouble condition, such as jamming of one of the motors, the signal at pin 926 changes, causing the controller to set a trouble flag. When the trouble flag is set, the controller activates the buzzer 123 such that it produces a beep twice per second for thirty seconds.

The controller 254 monitors the supply voltage (which in the exemplary embodiment shown is provided by the battery 258) through pin 930. If the controller senses a voltage less than a predetermined threshold, e.g., 11.5 volts, the LED indicator 122 is flashed and a signal is provided on a pin 934 to drive the buzzer 123. In one embodiment, the controller 254 is programmed to activate the buzzer 123 once per second for thirty seconds.

The controller drives the ozone generator 38 through pins 938 and 942. In the particular embodiment shown, each pin outputs a 20 kHz square wave for five minutes. The square wave signals are conditioned and amplified through drive circuits 950 and 952 which have two output stage devices 954 and 956, respectively. In the embodiment shown, the devices 954 and 956 are field effect transistors. The devices 954 and 956 are coupled to the transformer 217, which steps up the voltage of the signal provided to the transformer 217 to provide the 3000 V signal required to operate the ozone generator 38. The operation of the devices 954 and 956 are monitored through pin 958. If a trouble condition is sensed at the pin 958, the trouble flag is set and the controller 254 activates the buzzer 123, in the manner described above.

In general, it will be appreciated that the container 14 and articles 50 therein (or just the articles 50 therein) can be agitated in a number of different manners by a number of different driving devices, each of which falls within the spirit and scope of the present invention. As used herein and in the appended claims, such driving devices are referred to simply as “motors”. Accordingly, the term “motor” as used herein and in the appended claims is understood to encompass all driving and actuating elements capable of moving the container 14, including without limitation electric and hydraulic motors, pneumatic and hydraulic cylinders, solenoids, and the like.

1. An article sanitation device comprising:
a container defining an inner chamber and including an inlet and an outlet;
a motor operable to move the container;
a duct in fluid communication with the inlet and the outlet;
a fan operable to circulate fluid along a flow path that extends through the inner chamber and the duct;
an ozone generator operable to provide ozone to the inner chamber;
an ozone neutralizer operable to remove ozone from the inner chamber; and
a filter element positioned to remove particulate matter from the fluid in the flow path, the ozone and the filter element cooperating to substantially destroy biological contaminates in the inner chamber and remove the biological contaminates and particulate matter from the flow path.

2. The article sanitation device of claim 1, wherein the ozone generator supplies ozone to the flow path upstream of the inlet.

3. The article sanitation device of claim 1, wherein the ozone neutralizer removes ozone from the flow path downstream of the outlet.

4. The article sanitation device of claim 1, wherein the filter element is in the flow path between the inner chamber and the ozone neutralizer.

5. The article sanitation device of claim 1, further comprising a housing surrounding the container, wherein the container is rotatable within the housing by the motor to agitate an article to be sanitized inserted into the inner chamber.

6. The article sanitation device of claim 5, wherein the container includes an access opening through which the article to be sanitized is insertable into the inner chamber, the article sanitation device further comprising a lid coupled to the housing and movable to open and close the container.

7. The article sanitation device of claim 6, wherein a portion of the duct extends through the lid when the container is closed.

8. The article sanitation device of claim 5, further comprising an outer chamber surrounding the inner chamber and defined between the container and the housing, wherein the duct extends through the outer chamber.

9. The article sanitation device of claim 5, further comprising an indicator coupled to the housing, the indicator indicating a condition of sanitation of the article to be sanitized.

10. The article sanitation device of claim 5, wherein the housing at least partially defines a mailbox.

11. A mailbox for destroying contaminants of and removing particulate matter from mail, the mailbox comprising:
an inner container defining an inner chamber having an inlet and an outlet;
a fan operable to circulate fluid through the inner chamber;
an ozone generator operable to provide ozone to the inner chamber to destroy contaminants; and
a filter element positioned to remove particulate matter from the inner chamber.

12. The mailbox of claim 11, further comprising an outer container at least partially defining an outer chamber having an access opening, wherein the inner container is inside the outer container.

13. The mailbox of claim 12, wherein the inlet is located at the access opening.

14. The mailbox of claim 12, wherein the inner container is rotatable with respect to the outer container to agitate the mail.

15. The mailbox of claim 11, further comprising a duct communicating with the inlet and the outlet, wherein the ozone generator is coupled to the duct.

16. The mailbox of claim 15, wherein the filter element is in the duct downstream of the outlet.

17. The mailbox of claim 15, further comprising an ozone neutralizer coupled to the duct to remove ozone from the duct.

18. A method for sanitizing mail, the method comprising:
providing a mailbox including an inner chamber and a selectively closable access opening;
inserting an article of mail into the inner chamber;
closing the access opening;
generating a flow of fluid including increased levels of ozone through the inner chamber upon closing of the access opening;
filtering the flow of fluid to remove particulate matter from the flow of fluid; and
indicating that the mail has been sanitized.

19. The method of claim 18, further comprising removing the ozone from the flow of fluid.

20. The method of claim 18, further comprising:
removing the sanitized mail from the mailbox;
receiving a signal that indicates the sanitized mail has been removed; and
disabling the mailbox in response to receiving the signal, wherein disabling the mailbox prevents the act of generating a flow of fluid upon a subsequent closing of the access opening.

21. The method of claim 18, further comprising rotating the inner chamber upon closing of the access opening.

22. The method of claim 18, further comprising:
providing an electronic controller including a timer;
starting the timer upon closing the access opening; and
stopping the generation of the flow of fluid after a period of time is counted by the timer.

23. The method of claim 22, wherein the act of generating a flow of fluid includes introducing additional ozone into the flow of fluid, the method further comprising stopping the introduction of additional ozone prior to the counting of the period of time by the timer.

24. A method for sanitizing an article comprising:
providing a container defining an inner chamber and having a selectively closable access opening;
inserting an article to be sanitized into the inner chamber;
closing the access opening;
generating a flow of fluid through the inner chamber;
moving the container to agitate the article in the inner chamber; and
adding ozone to the flow of fluid to substantially destroy biological contaminates in the inner chamber.

25. The method of claim 24, further comprising filtering the flow of fluid to remove contaminates from the flow of fluid.

26. The method of claim 24, further comprising neutralizing ozone in the flow of fluid after adding ozone to the flow of fluid.

27. The method of claim 24, further comprising:
indicating that a sanitizing process performed on the article has been completed;
removing the sanitized article from the container;
receiving a signal indicating that the sanitized article has been removed; and
disabling future execution of the generating, moving, and adding steps in response to receiving the signal.

28. The method of claim 24, wherein moving the container comprises rotating the container.

29. The method of claim 28, further comprising:
driving the container with a motor connected to an electrical power supply;
determining a power level of the electrical power supply;
comparing the power level to a lower limit; and
indicating when the power level is below the lower limit.

30. The method of claim 24, further comprising:
providing an electronic controller including a timer;
starting the timer after closing the access opening; and
stopping generation of the flow of fluid after a period of time determined by the timer.

31. The method of claim 30, further comprising stopping adding of ozone to the flow of fluid prior to stopping generation of the flow of fluid.
